(19) United States
(12) Patent Application Publication
(10) Pub. No.: US 2011/0256237 A1
(43) Pub. Date: Oct. 20, 2011

(54) METHODS AND COMPOSITIONS FOR TREATING ACNE VULGARIS AND ACNE ROSACEA

(75) Inventor: Joel E. Bernstein, Deerfield, IL (US)

(73) Assignee: ELORAC, INC., Vernon Hills, IL (US)

(21) Appl. No.: 12/761,953

(22) Filed: Apr. 16, 2010

(51) Int. Cl. A61K 33/34; A61P 17/10; A61K 31/519; A61K 33/30; A61K 31/4406; A61K 31/4415

(52) U.S. Cl. 424/630; 514/356; 514/348; 514/249; 424/641

(57) ABSTRACT

Methods and compositions are described for treating acne vulgaris and acne rosacea by administering compositions suitable for oral administration. Suitable compositions contain high dosages of the vitamin nicotinamide combined with much lower dosages of azelaic acid.

### BACKGROUND

Acne vulgaris is a disease of the pilosebaceous glands and is characterized by an unsightly eruption of the skin of the face, neck, back and chest. Acne vulgaris is a common affliction of the adolescent and also affects a small but significant percentage of the adult population. Acne vulgaris lesions are of four basic types: comedones (blackheads or whiteheads), papules, pustules, and cysts (or nodules). Acne vulgaris involvement results in unsightly lesions, particularly on the face, and in some cases resulting in severe scarring. Various topical agents used in the treatment of acne vulgaris include sulfur, resorcinol, salicylic acid, benzoyl peroxide, various retinoids including tretinoin, tazarotine and adapalene, carbamide peroxide, azelaic acid, and topical antibiotics.

Acne rosacea, commonly called simply rosacea, is an inflammatory disorder of the skin that, despite its name, seems to bear no relationship to acne vulgaris. In contrast to acne vulgaris, rosacea occurs predominantly in middle-aged adults and is virtually never observed in adolescents or young adults. Rosacea is characterized by inflammatory lesions of the skin that resemble acne vulgaris papules and pustules (“acneform” lesions) and a disorder of the superficial cutaneous vasculature resulting in erythema, accentuated flushing and telangiectasia. Comedones, a hallmark of acne vulgaris, do not occur as part of the rosacea “complex.” Rosacea is treated with a variety of topical therapies including sodium sulfacetamide, topical antibiotics, azelaic acid and metronidazole.

Apart from the topical acne-treatment agents mentioned above, orally administered agents are frequently utilized to treat acne vulgaris, and much less frequently used to treat acne rosacea. Such oral treatments are most commonly oral antibiotics such as tetracycline, Minocycline or doxycycline or oral retinoids, specifically isotretinoin. Such oral treatments, while generally more effective than topical therapies, are unfortunately not infrequently accompanied by serious systemic side effects ranging from diarrhea and yeast infections with oral antibiotics to depression and inflammatory bowel disease with isotretinoin.

In an effort to successfully treat acne vulgaris and rosacea, without risk of serious systemic side effects, nutritional supplements containing high dosages of nicotinamide (also known as niacinamide) have been employed. Although such supplements provide some clinical benefits, they are not as effective as the oral antibiotics or retinoids.

### SUMMARY

Surprisingly, by combining a high dosage of nicotinamide with a relatively low dosage of azelaic acid in a tablet capsule or suspension for oral ingestion, and administering such to patients with acne vulgaris or rosacea, treatment results comparable to or better than oral antibiotics are produced without any serious systemic side effects.

Methods and compositions are described for treating acne vulgaris and acne rosacea by administering compositions suitable for oral administration. Suitable compositions contain high dosages of the vitamin nicotinamide combined with much lower dosages of azelaic acid. By combining nicotinamide and azelaic acid and administering such formulations in capsules, tablets or oral suspensions, one can achieve dramatic improvement in patients with acne vulgaris or acne rosacea, with such improvement comparable to that seen with oral antibiotics. These compositions, consequently, have the advantage over oral antibiotics of producing substantial improvement in these skin diseases without producing any systemic side effects.

### DETAILED DESCRIPTION

Novel and improved methods and compositions for treating acne vulgaris and acne rosacea are described. High dosages of nicotinamide (aka niacinamide) ranging from 100 mg to 1000 mg per unit dose and up to 3000 mg daily were combined in formulations suitable for oral administration with much lower dosages of azelaic acid, the azelaic acid dosage ranging from 1 mg to 50 mg per unit dosage and up to 150 mg daily.

Formulations suitable for oral administration include tablets, capsules, caplets, and suspensions. Such oral formulations optionally include beside nicotinamide and azelaic acid, other vitamins, minerals, and nutritional ingredients, including but not limited to folic acid, pyridoxine, vitamin A, vitamin E, vitamin C, vitamin D, zinc, copper, glycyrrhizinic acid, glycyrrhetinic acid, carbenoxolone, resveratrol, and beta carotene.

Formulations suitable for oral administration containing 100 mg to 1000 mg per unit dose of nicotinamide and also containing azelaic acid from 1 mg to 50 mg per unit dose are administered once to four times daily as tablets, capsules, caplets or suspensions to patients with either acne vulgaris or rosacea. Such daily therapy may be provided for treatment durations of as little as 1 to 2 weeks or continuously for durations of up to several years or more.

### EXAMPLES

The following examples are not meant to be limiting. It is possible to produce still other embodiments without departing from the concepts of the materials and methods claimed herein. Further embodiments are within the ability of one skilled in the art.

### Example 1

Oral tablets containing 600 mg nicotinamide, 5 mg azelaic acid, 10 mg zinc (as zinc oxide), 5 mg pyridoxine, 1.5 mg copper (as cupric oxide), and 500 mcg folic acid are administered to patients with mild to moderate inflammatory acne vulgaris once to four times daily, with a majority of such patients expected to demonstrate significant reductions in their acne lesions over treatment periods of 1 to 52 weeks.

### Example 2

Oral tablets containing 750 mg nicotinamide, 50 mg azelaic acid, 1 mg folic acid, 20 IU vitamin A, 100 IU vitamin D, and pyridoxine 5 mg, are administered to patients with acne vulgaris once or twice daily for 12 consecutive weeks. Such patients are expected to show dramatic improvement in their acne conditions by the end of the 12-week administration period.

### Example 3

Gelatin capsules containing 100 mg nicotinamide, 1 mg azelaic acid, 500 mcg folic acid, 100 IU vitamin A, 25 mg zinc oxide, 1.5 mg copper oxide, and 2 mg glycyrrhetinic acid are administered four times daily to patients with rosacea for from 4 up to 52 weeks of continuous therapy. Acneform lesions of rosacea are expected to decrease significantly over this treatment course without any of the systemic side effects associated with oral antibiotics.

### Example 4

Gelatin capsules containing 600 mg nicotinamide, 50 mg azelaic acid, 10 mg zinc oxide, 10 mg pyridoxine, 500 mg folic acid, 20 mg beta carotene and 1 mg carbenoxolone administered two capsules twice daily are expected to produce impressive clinical improvement in the complexions of patients with severe inflammatory acne vulgaris.

### Example 5

Caplets containing 250 mg nicotinamide, 5 mg azelaic acid, 100 IU vitamin A and 500 mcg folic acid are administered to patients with rosacea with the expected result that such patients will have marked improvement in their acneform lesions without any systemic side effects.

### Example 6

An oral suspension flavored with artificial or natural fruit flavors (e.g. cherry, orange, pineapple) containing 150 mg nicotinamide and 5 mg azelaic acid in each 5 ml of liquid is administered to adolescent males or females with inflammatory acne vulgaris in a dosage of 1 teaspoonful (5 ml) twice daily with resulting excellent control of acne and no serious side effects.

1. A method of treating acne vulgaris and acne rosacea, the method comprising administering pharmaceutical formulations suitable for oral administration wherein the formulations contain high dosages of nicotinamide combined with much lower dosages of azelaic acid.

2. The method of claim 1, wherein high dosages of nicotinamide are in a range of about 100 mg to 1000 mg per unit dosage of the formulations and lower dosages of azelaic acid are present in a range of about 1 mg to 50 mg per unit dosage of the formulations.

3. The method of claim 1, wherein the pharmaceutical formulations suitable for oral administration are administered in a form selected from the group consisting of tablets, capsules, caplets, and suspensions.

4. The method of claim 1, wherein the oral formulations optionally contain pharmaceutical excipients, and other nutritional agents and vitamins.

5. The method of claim 4, wherein the other nutritional and vitamin ingredients are selected from the group consisting of folic acid, pyridoxine, vitamin A, beta carotene, vitamin E, vitamin D, vitamin C, zinc, copper, glycyrrhizinic acid, glycyrrhetinic acid, carbenoxolone, and resveratrol.

6. A composition for treating acne vulgaris or acne rosacea, the composition comprising a pharmaceutical preparation suitable for oral administration wherein nicotinamide and azelaic acid are present together.

7. The composition of claim 6, wherein the nicotinamide is present in a dosage of about 100 mg to 1000 mg and the azelaic acid is present in a dosage of about 1 mg to 50 mg.

8. The composition of claim 6, wherein the amount of nicotinamide present is always at least 5 times the amount of azelaic acid present in the composition.

9. The composition of claim 6, wherein the pharmaceutical preparations suitable for oral administration are in a form selected from the group consisting of tablets, capsules, caplets, and suspensions.

10. The composition of claim 6, wherein the compositions suitable for oral administration optionally include pharmaceutical excipients, nutritional agents and vitamins.

11. The composition of claim 10, wherein the other nutritional or vitamin ingredients are selected from the group consisting of folic acid, pyridoxine, vitamin A, beta carotene, vitamin E, vitamin C, vitamin D, zinc, copper, glycyrrhizinic acid, glycyrrhetinic acid, carbenoxolone, and resveratrol.
